(19) United States
(12) Patent Application Publication
(10) Pub. No.: US 2006/0159712 A1
(43) Pub. Date: Jul. 20, 2006

(54) Lipid particles comprising bioactive agents, methods of preparing and uses thereof

(75) Inventors: Jin K. Lee, Belle Mead, NJ (US); RoseAnn Kurumunda, Plainsboro, NJ (US)

Correspondence Address: FOLEY HOAG, LLP;PATENT GROUP, WORLD TRADE CENTER WEST, 155 SEAPORT BLVD, BOSTON, MA, 02110, US

(73) Assignee: Transave, Inc., Monmouth Junction, NJ (US)

(21) Appl. No.: 11/302,455

(22) Filed: Dec. 13, 2005

Related U.S. Application Data
(60) Provisional application No. 60/635,832, filed on Dec. 14, 2004.

(51) Int. Cl. A61K 9/00; A61K 31/28; A61K 33/24

(52) U.S. Cl. 424/400; 424/649; 514/283; 514/492

(57) ABSTRACT

The present invention relates to a non-liposomal lipid particle comprising an amphiphile-coated complex of a hydrophobic bioactive agent and an inverted hexagonal phase forming lipid, and methods of preparing and kits thereof.

### RELATED APPLICATIONS

This application claims the benefit of priority to U.S. Provisional Patent Application Ser. No. 60/635,832, filed Dec. 14, 2004.

### BACKGROUND OF THE INVENTION

Lipid particle complexes have been long recognized as drug delivery systems which can improve therapeutic and diagnostic effectiveness of many bioactive agents and contrast agents. Experiments with a number of different antibiotics and X-ray contrast agents have shown that better therapeutic activity or better contrast with a higher level of safety can be achieved by encapsulating bioactive agents and contrast agents with lipid complexes.

Essentially, there have to date been three major particulate lipid-water systems which have been considered as suitable for drug delivery, namely such based on the lamellar mesophase as liposomes, micellar-based phases including micelles, reversed micelles, and mixed micelles and various kinds of emulsions including microemulsions, as well as more novel carriers as ISCOM's (Morein 1988) (a general text concerning these systems is Pharmaceutical Dosage Forms, Disperse Systems 1988). The latter system has been utilized for intravenous nutrition since the beginning of this century and as an adjuvant system known as the Freunds adjuvant. These are of oil-in-water (O/W) and water-in-oil (W/O) types, respectively. Liposomes have since their discovery been extensively investigated as drug delivery systems for various routes and drugs. The development of new colloidal drug carrier systems is a research area of intensive activity and it is likely that new systems, especially new emulsion based systems, will appear in the near future. Lipid-based vehicles can take several different morphological forms such as normal and reversed micelles, microemulsions, liposomes including variants as unilamellar, multilamellar, etc., emulsions including various types as oil-in-water, water-in-oil, multiple emulsions, etc., suspensions, and solid crystalline. In addition so called niosomes formed from nonionic surfactants have been investigated as a drug vehicle. The use of these vehicles in the field of drug delivery and biotechnology is well documented (Mulley 1974, Davis et al. 1983, Gregoriadis 1988a, Liebermann et al 1989). Particularly in the field of drug delivery the use of lipid-based drug delivery systems, especially dispersed systems, has attained increasing interest as the pharmaceutical industry is developing more potent and specific-and thus more cytotoxic-drugs.

Liposomes can be produced by a variety of methods (for a review, see, e.g., Cullis et al. (1987)). Bangham's procedure (J. Mol. Biol. (1965)) produces ordinary multilamellar vesicles (MLVs). Lenk et al. (U.S. Pat. Nos. 4,522,803, 5,030,453 and 5,169,637), Fountain et al. (U.S. Pat. No. 4,588,578) and Cullis et al. (U.S. Pat. No. 4,975,282) disclose methods for producing multilamellar liposomes having substantially equal interlamellar solute distribution in each of their aqueous compartments. Paphadjopoulos et al., U.S. Pat. No. 4,235,871, discloses preparation of oligolamellar liposomes by reverse phase evaporation.

Unilamellar vesicles can be produced from MLVs by a number of techniques, for example, the extrusion of Cullis et al. (U.S. Pat. No. 5,008,050) and Loughrey et al. (U.S. Pat. No. 5,059,421)). Sonication and homogenization can be so used to produce smaller unilamellar liposomes from larger liposomes (see, for example, Paphadjopoulos et al. (1968); Deamer and Uster (1983); and Chapman et al. (1968)).

The original liposome preparation of Bangham et al. (J. Mol. Biol., 1965, 13:238-252) involves suspending phospholipids in an organic solvent, which is then evaporated to dryness leaving a phospholipid film on the reaction vessel. Next, an appropriate amount of aqueous phase is added, the mixture is allowed to “swell”, and the resulting liposomes which consist of multilamellar vesicles (MLVs) are dispersed by mechanical means. This preparation provides the basis for the development of the small sonicated unilamellar vesicles described by Papahadjopoulos et al. (Biochim. Biophys, Acta., 1967, 135:624-638), and large unilamellar vesicles.

Techniques for producing large unilamellar vesicles (LUVs), such as, reverse phase evaporation, infusion procedures, and detergent dilution, can be used to produce liposomes. A review of these and other methods for producing liposomes may be found in the text Liposomes, Marc Ostro, ed., Marcel Dekker, Inc., New York, 1983, Chapter 1, the pertinent portions of which are incorporated herein by reference. See also Szoka, Jr. et al., (1980, Ann. Rev. Biophys. Bioeng., 9:467), the pertinent portions of which are also incorporated herein by reference.

Other techniques that are used to prepare vesicles include those that form reverse-phase evaporation vesicles (REV), Papahadjopoulos et al., U.S. Pat. No. 4,235,871. Another class of liposomes that may be used is characterized as having substantially equal lamellar solute distribution. This class of liposomes is denominated as stable plurilamellar vesicles (SPLV) as defined in U.S. Pat. No. 4,522,803 to Lenk, et al. and includes monophasic vesicles as described in U.S. Pat. No. 4,588,578 to Fountain, et al. and frozen and thawed multilamellar vesicles (FATMLV) as described above.

A variety of sterols and their water soluble derivatives such as cholesterol hemisuccinate have been used to form liposomes; see specifically Janoff et al., U.S. Pat. No. 4,721,612, issued Jan. 26, 1988, entitled “Steroidal Liposomes.” Mayhew et al., PCT Publication No. WO 85/00968, published Mar. 14, 1985, described a method for reducing the toxicity of drugs by encapsulating them in liposomes comprising alpha-tocopherol and certain derivatives thereof. Also, a variety of tocopherols and their water soluble derivatives have been used to form liposomes, see Janoff et al., PCT Publication No. 87/02219, published Apr. 23, 1987, entitled “Alpha Tocopherol-Based Vesicles”.

In a liposome-drug delivery system, a bioactive agent such as a drug is entrapped in the liposome and then administered to the patient to be treated. For example, see Rahman et al., U.S. Pat. No. 3,993,754; Sears, U.S. Pat. No. 4,145,410; Paphadjopoulos et al., U.S. Pat. No. 4,235,871; Schneider, U.S. Pat. No. 4,224,179; Lenk et al., U.S. Pat. No. 4,522,803; and Fountain et al., U.S. Pat. No. 4,588,578. Alternatively, if the bioactive agent is lipophilic, it may associate with the lipid bilayer.

Although liposomal lipid complexes have been extensively studied for drug delivery systems, non-liposomal lipid complexes have received less attention. Such non-liposomal lipid complexes are characterized, for example, by: (1) freeze-fracture electron micrographs (Deamer et al., Biochim. Biophys. Acta, 1970, 219:47-60), demonstrating non-liposomal complexes; (2) captured volume measurements (Deamer et al., Chem. Phys. Lipids, 1986, 40:167-188), demonstrating essentially zero entrapped volumes and therefore being non-liposomal; (3) differential scanning calorimetry (DSC) (Chapman, D., in: Liposome Technology, Gregoriadis, G., ed., 1984, CRC Press, Boca Raton), showing no lipid bilayer pre-transition phase or main transition; (4) ³¹P-NMR spectra (Cullis et al., 1982 in: Membrane Fluidity in Biology, Academic Press, Inc., London & N.Y.), suggesting characteristics of highly immobilized lipid (broad isotropic); and (5) x-ray diffraction data (Shipley et al., in: Biomembranes, 1973, Chapman, D. and Wallach, D., eds., Vol 2: 1, Academic Press, Inc., London & N.Y.), indicative of gel phase lipid. Also characteristic of these systems is the complete association of the drug with the lipid as evidenced by density gradient centrifugation. In this technique the gradient is centrifuged at an elevated force (about 230,000×g) for about 24 hours. This insures that all the components in the gradient reach their equilibrium density positions. Elution profiles of these systems show overlapping drug and lipid peaks, which indicates all of the drug is associated with the lipid.

Hydrophobic drugs are generally difficult to load into conventional phospholipid liposomes because they tend to crystallize rather than incorporate into the phospholipid liposomal membrane. Thus, non-liposomal drug-delivery systems have been a more promising way of formulating a hydrophobic drug.

U.S. Pat. No. 6,406,713 discloses high drug to lipid complexes (HDLC) that are non-liposomal when they employ 25 mole percent to about 50 mole percent of drug. However, even higher drug to lipid ratios would be beneficial.

U.S. Pat. No. 5,531,925 discloses non-liposomal particles having an interior non-lamellar lyotropic liquid crystalline phase selected from reversed cubic liquid crystalline phase, reversed hexagonal liquid crystalline phase, or a homogeneous L3 phase; and a surface phase selected from a lamellar crystalline phase, a lamellar liquid crystalline phase, or an L3 phase.

New forms of lipid particles with new properties that can accommodate higher drug loading levels and exhibit favorable delivery profiles are needed.

### SUMMARY OF THE INVENTION

In part, the present invention features a lipid particle comprising an amphiphile-coated complex of a hydrophobic bioactive agent and an inverted hexagonal phase-forming lipid. Preferred hydrophobic bioactive agents include taxanes such as paclitaxel, other cancer treating compounds such as amphotericin B, camptothecin, and platinum compounds such as cisplatin.

Preferred inverted hexagonal phase-forming lipids include phosphatidylethanolamines (PE), such as dioleoylphosphatidylethanolamine (DOPE), dimyristooylphosphatidylethanolamine (DMPE), or dipalmitoylphophatidylethanolamine (DPPE).

Preferred amphiphiles include phosphatidylcholine (PC), phosphatidylglycerol (PG), phosphatidylserine (PS), phosphatidylethanolamine (PE), phosphatidylinositol (PI), phosphoric acid (PA), sphingomyelin, ganglioside, lysoPC, PEG-lipids, surfactants, or combinations thereof.

In part, the present invention features methods of preparing the lipid particles as well as a method of treating a patient for a condition or disease comprising administering to the patient a therapeutically effective amount of the lipid particles, which include a hydrophobic bioactive agent that is useful for treating the disease or condition.

Preferred methods of preparing the lipid particles of the present invention include sonicating a mixture of the hydrophobic bioactive agent and the inverted hexagonal phase forming lipid in deionized water followed by the addition of the amphiphile and further sonicating until a milky suspension forms. In a further embodiment, the resulting lipid particles may be fractionated to obtain particles of certain parameters.

In another embodiment, the lipid particles of the present invention can by formed by an infusion process. In this process the hydrophobic bioactive agent and the inverted hexagonal phase-forming lipid are codissolved in a non-aqueous solvent and infused into an aqueous solution followed by removal of the non-aqueous solvent. The amphiphile is dissolved in a non-aqueous solvent and infused in an aqueous solution, followed by removal of the non-aqueous solvent. These two suspensions prepared separately are mixed together and sonicated. In a further embodiment, the resulting lipid particles may be fractionated to obtain particles of certain parameters.

In part, the present invention features a kit comprising the lipid particles of the present invention and instructions for use thereof.

These embodiments of the present invention, other embodiments, and their features and characteristics, will be apparent from the description, drawings and claims that follow.

### BRIEF DESCRIPTION OF THE DRAWINGS

FIG. 1 depicts the clearance of paclitaxel in rat lungs after intratracheal instillation of the lipid particles with paclitaxel vs. taxol (cremophore formulation, micellar). Female Sprague/Dawley rats were given the lipid particles with paclitaxel (13.7 mg/kg)/taxol (cremophore formulation, 6 mg/kg) by intratracheal instillation. Rats were sacrificed after 0, 1, 2, 6, 24, 48 hrs and the paclitaxel level in lung was determined by HPLC. Data for taxol were normalized to the dose of the lipid particles with paclitaxel.

FIG. 2 depicts the structure of bioactive agent containing lipid particles of the present invention: A) depicts the normal reverse hexagonal(II) phase of PE, B) depicts paclitaxel dissolved in the hydrocarbon region of the reverse hexagonal(II) phase of PE, and C) the amphiphile stabilized paclitaxel containing lipid particle sized by sonication.

FIG. 3 depicts a freeze-facture EM image of paclitaxel containing lipid particles of the present invention. The white bar represents 1 micron.

### DETAILED DESCRIPTION OF THE INVENTION

### DEFINITIONS

For convenience, before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

The articles “a” and “an” are used herein to refer to one or to more than one (i.e. to at least one) of the grammatical object of the article. By way of example, “an element” means one element or more than one element.

The term “amphiphile” is used herein to mean any substance containing both polar, water-soluble groups and non-polar, water-insoluble groups.

The term “bioavailable” is art-recognized and refers to a form of the subject invention that allows for it, or a portion of the amount administered, to be absorbed by, incorporated to, or otherwise physiologically available to a subject or patient to whom it is administered.

The terms “comprise” and “comprising” are used in the inclusive, open sense, meaning that additional elements may be included.

The term “hydrophobic bioactive agent” as used herein refers to any bioactive agent that under the reaction conditions of its medium has low solubility in a polar solvent such as water. Examples of reaction conditions include pH, temperature, and concentration. Therefore, hydrophobic agents may include agents that may have a high solubility under certain pHs or temperatures, but under the pHs or temperatures being used have a low solubility. Non-limiting examples of a hydrophobic bioactive agent include platinum complexes under the reaction conditions used herein.

The term “including” is used herein to mean “including but not limited to”. “Including” and “including but not limited to” are used interchangeably.

The phrase “inverted hexagonal phase forming lipid” is used herein to mean any lipid capable of forming an inverted hexagonal crystal phase. Generally, phospholipids are capable of forming an inverted hexagonal phase. Although some phosphatidylglycerols (PG), phosphatidylacids (PA), and phosphatidylserines (PS) can form inverted hexagonal phases under high temperatures (>95 ° C.), phosphatidylethanolamines (PE), such as for example, dioleylphosphatidylethanolamine (DOPE), form an inverted hexagonal phase under more general room temperature conditions. In one embodiment, inverted hexagonal phase forming lipids refers to lipids capable of forming an inverted hexagonal phase at room temperature. These lipids will have a phase transition temperature (i.e. the temperature at which a transition from lamellar phase to inverted hexagonal phase may occur) that is below room temperature. In another embodiment, the inverted hexagonal phase forming lipid comprises a fatty acid chain.

A “patient,” “subject” or “host” may be a human or non-human animal.

The term “pharmaceutically acceptable salts” is art-recognized and refers to the relatively non-toxic, inorganic and organic acid addition salts of compounds, including, for example, those contained in compositions of the present invention.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof from one organ, or portion of the body, to another organ, or portion of the body. Each carrier must be acceptable in the sense of being compatible with the subject composition and its components and not injurious to the patient. Some examples of materials which may serve as pharmaceutically acceptable excipients include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “prophylactic” or “therapeutic” treatment is art-recognized and refers to administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

The phrase “therapeutic effect” is art-recognized and refers to a local or systemic effect in animals, particularly mammals, and more particularly humans caused by a pharmacologically active substance. The term thus means any substance intended for use in the diagnosis, cure, mitigation, treatment or prevention of disease or in the enhancement of desirable physical or mental development and/or conditions in an animal or human. The phrase “therapeutically-effective amount” means that amount of such a substance that produces some desired local or systemic effect at a reasonable benefit/risk ratio applicable to any treatment. The therapeutically effective amount of such substance will vary depending upon the subject and disease condition being treated, the weight and age of the subject, the severity of the disease condition, the manner of administration and the like, which can readily be determined by one of ordinary skill in the art.

The term “treating” is art-recognized and refers to curing as well as ameliorating at least one symptom of any condition or disease.

The definitions above are read in light of the remainder of the disclosure and understood as by a person of skill in the art. They are not meant to limit any contemplated equivalents. Contemplated equivalents of the lipid particles, subunits and other compositions described above include such materials which otherwise correspond thereto, and which have the same general properties thereof (e.g., biocompatible), wherein one or more simple variations of substituents are made which do not adversely affect the efficacy of such molecule to achieve its intended purpose. In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants which are in themselves known, but are not mentioned here.

Hydrophobic Bioactive Agent

The hydrophobic bioactive agent plays a unique role in the lipid particle delivery systems disclosed herein. Its presence is needed for the formation of the lipid particle. Attempts to make placebo lipid particles in the absence of the hydrophobic bioactive agent were not successful. It is believed that the hydrophobic bioactive agent complexes with the hydrophobic portion of an inverted hexagonal phase-forming lipid, resulting in a structure that allows formation of the lipid particles disclosed herein in the presence of an amphiphile.

The hydrophobic bioactive agent may be any bioactive agent that has low solubility in an aqueous environment under the reaction conditions used. Some specific examples of hydrophobic bioactive agents that can be present in the compositions and the uses of the composition in the treatment of disease include: sulfonamide, such as sulfonamide, sulfamethoxazole and sulfacetamide; trimethoprim, particularly in combination with sulfamethoxazole; a quinoline such as norfloxacin and ciprofloxacin; a beta-lactam compound including a penicillin such as penicillin G, penicillin V, ampicillin, amoxicillin, and piperacillin, a cephalosporin such as cephalosporin C, cephalothin, cefoxitin and ceftazidime, other beta-lactarn antibiotics such as imipenem, and aztreonam; a beta lactamase inhibitor such as clavulanic acid; an aminoglycoside such as gentamycin, amikacin, tobramycin, neomycin, kanamycin and netilmicin; a tetracycine such as chlortetracycline and doxycycline; chloramphenicol; a macrolide such as erythromycin; or miscellaneous antibiotics such as clindamycin, a polymyxin, and bacitracin for anti-bacterial, and in some cases antifungal, infections; a polyene antibiotic such as amphotericin B, nystatin, and hamycin; flucytosine; an imidazole or a triazole such as ketoconazole, miconazole, itraconazole and fluconazole; griseofulvin for anti-Fungal diseases such as aspergillosis, candidaisis or histoplasmosis; zidovudine, acyclovir, ganciclovir, vidarabine, idoxuridine, trifluridine, an interferon (e.g, interferon alpha-2a or interferon alpha-2b) and ribavirin for anti-viral disease; aspirin, phenylbutazone, phenacetin, acetaminophen, ibuprofen, indomethacin, sulindac, piroxicam, diclofenac; gold and steroidal anti-inflammatories for inflammatory diseases such as arthritis; an ACE inhibitor such as captopril, enalapril, and lisinopril; the organo nitrates such as amyl nitrite, nitroglycerin and isosorbide dinitrate; the calcium channel blockers such as diltiazem, nifedipine and verapamil; the beta adrenegic antagonists such as propranolol for cardiovascular disease; a diuretic such as a thiazide; e.g., benzothiadiazine or a loop diuretic such as furosemnide; a sympatholytic agent such as methyldopa, clonidine, gunabenz, guanaethidine and reserpine; a vasodilator such as hydalazine and minoxidil; a calcium channel blocker such as verapimil; an ACE inhibitor such as captopril for the treatment of hypertension; quinidine, procainamide, lidocaine, encainide, propranolol, esmolol, bretylium, verapimil and diltiazem for the treatment of cardiac arrhythmia; lovostatin, lipitor, clofibrate, cholestryamine, probucol, and nicotinic acid for the treatment of hypolipoproteinernias; an anthracycline such as doxorubicin, daunorubicin and idarubicin; a covalent DNA binding compound, a covalent DNA binding compound and a platinum compound such as cisplatin and carboplatin; a folate antagonist such as methotrexate and trimetrexate; an antimetabolite and a pyrimidine antagonist such as fluorouracil, 5-fluorouracil and fluorodeoxyuridine; an antimetabolite and a purine antagonist such as mercaptopurine, 6-mercaptopurine and thioguanine; an antimetabolite and a sugar modified analog such as cytarabine and fludarabine; an antimetabolite and a ribonucleotide reductase inhibitor such as hydoxyurea; a covalent DNA binding compound and a nitrogen mustard compound such as cyclophosphamide and ifosfamide; a covalent DNA binding compound and an alkane sulfonate such as busulfane; a nitrosourea such as carmustine; a covalent DNA binding compound and a methylating agent such as procarbazine; a covalent DNA binding compound and an aziridine such as mitomycin; a non covalent DNA binding compound; a non covalent DNA binding compound such as mitoxantrone and, bleomycin; an inhibitor of chromatin function and a topoisomerase inhibitor such as etoposide, teniposide, camptothecin and topotecan; an inhibitor of chromatin function and a microtubule inhibitor such as the vinca alkaloids including vincristine, vinblastin, vindisine, and paclitaxel, taxotere or another taxane; a compound affecting endocrine function such as prednisone, prednisolone, tamoxifen, leuprolide, ethinyl estradiol, an antibody such as herceptin; a gene such as the p-53 gene, the p 16 gene, the MIT gene, and the gene E-cadherin; a cytokine such as the interleukins, particularly, IL-1, IL-2, IL-4, IL-6, IL-8 and IL-12, the tumor necrosis factors such as tumor necrosis factor-alpha and tumor necrosis factor-beta, the colony stimulating factors such as granulocyte colony stimulating factor (G-CSF), macrophage colony stimulating factor (M-CSF) and, granulocyte macrophage colony stimulating factor (GM-CSF) an interferon such as interferon-alpha, interferon-beta 1, interferon-beta 2, and interferon-gamma; all-trans retinoic acid or another retinoid for the treatment of cancer; an immunosupressive agent such as: cyclosporine, an immune globulin, and sulfasazine, methoxsalen and thalidoimide; insulin and glucogon for diabetes; calcitonin and sodium alendronate for treatment of osteoporosis, hypercalcemia and Paget's Disease; morphine and related opioids; meperidine or a congener; methadone or a congener; an opioid antagonist such as nalorphine; a centrally active antitussive agent such as dexthromethrophan; tetrahydrocannabinol or marinol, lidocaine and bupivicaine for pain management; chloropromazine, prochlorperazine; a cannabinoid such as tetrahydrocannabinol, a butyrophenone such as droperidol; a benzamide such as metoclopramide for the treatment of nausea and vomiting; heparin, coumarin, streptokinase, tissue plasminogen activator factor(t-PA) as anticoagulant, antithrombolytic or antiplatelet drugs; heparin, sulfasalazine, nicotine and adrenocortical steroids and tumor necrosis factor-alpha for the treatment of inflammatory bowel disease; nicotine for the treatment of smoking addiction; growth hormone, luetinizing hormone, corticotropin, and somatotropin for hormonal therapy; and adrenaline for general anaphylaxis.

Further hydrophobic bioactive agents that can be present in the compositions of the inhalation system and the uses of the system in the treatment of disease include: a methylxanthine such as theophylline; cromolyn; a beta-adrenginic agonist such as albuterol and tetrabutaline; a anticholinergic alkaloid such as atropine and ipatropium bromide; adrenocortical steroids such as predisone, beclomethasone and dexamethasone for asthma or inflammatory disease; the anti-bacterial and antifungal agents listed above for anti-bacterial and anti-fungal infections in patients with lung disease (these are the specific diseases listed above in what lung disease includes), in particular this includes the use of aminoglycosides (e.g., amikacin, tobramycin and gentamycin), polymyxins (e.g., polymyxin E, colistin), carboxycillin (ticarcillin) and monobactams for the treatment of gram-negative anti-bacterial infections, for example, in cystic fibrosis patients, for the treatment of gram negative infections of patients with tuberculosis, for the treatment of gram negative infections in patients with chronic bronchitis and bronchiectasis, and for the treatment of gram negative infections in generally immuno-compromised patients; the use of pentamidine for the treatment of patients (e.g., HIV/AIDS patients) with Pneumocytis carinii infections; the use of a polyene antibiotic such as amphotericin B, nyststin, and hamycin; flucytosine; an imidazole or a triazole such as ketoconazole, miconazole, itraconazole and fluconazole; griseofulvin for the treatment of such fungal infections as aspergillosis, candidiasis and histoplasmosis, particularly those originating or diseminating to the lungs; the use of the corticosteroids and other steroids as listed above, as well as nonsteroidal anti-inflammatory drugs for the treatment of anti-inflammatory conditions in patients with lung disease (these are the specific diseases listed above in what lung disease includes); DNase, amiloride, CFTRcDNA in the treatment of cystic fibrosis; alpha-1-antitrypsin and alpha-1-antitrypsin cDNA for the treatment of emphysema; an aminoglycoside such as amikacin, tobramycin or gentamycin, isoniazid, ethambutol, rifampin and its analogs for the treatment of tuberculosis or mycobacterium infections; ribavirin for the treatment of respiratory synctial virus; the use of the anticancer agents listed above for lung cancer in particular vinorelbine, cisplatin, carboplatin, and taxanes such as paclitaxel, and other taxanes, camptothecin, topotecin, and other camptothecins, herceptin, the p-53 gene and IL-2. In addition, pharmaceutical bioactive agents such as Tarceva and Iressa may also be used.

The hydrophobic bioactive agents may contain more than one bioactive agent (e.g., two bioactive agents for a synergistic effect). In one embodiment, the hydrophobic bioactive agent is a platinum based bioactive agent. In a further embodiment, the bioactive agent is paclitaxel.

Lipids

The lipids used in the lipid particles presently disclosed can be synthetic, semi-synthetic or naturally-occurring lipids, and typically include phospholipids and sterols. In terms of phosholipids, they could include such lipids as egg phosphatidylcholine (EPC), egg phosphatidylglycerol (EPG), egg phosphatidylinositol (EPI), egg phosphatidylserine (EPS), phosphatidylethanolamine (EPE), and phosphatidic acid (EPA); the soya counterparts, soy phosphatidylcholine (SPC); SPG, SPS, SPI, SPE, and SPA; the hydrogenated egg and soya counterparts (e.g., BEPC, HSPC), other phospholipids made up of ester linkages of fatty acids in the 2 and 3 of glycerol positions containing chains of 12 to 26 carbon atoms and different head groups in the 1 position of glycerol that include choline, glycerol, inositol, serine, ethanolamine, as well as the corresponding phosphatidic acids. The chains on these fatty acids can be saturated or unsaturated, and the phospholipid may be made up of fatty acids of different chain lengths and different degrees of unsaturation. In particular, the compositions of the formulations can include dipalmitoylphosphatidylcholine (DPPC), a major constituent of naturally-occurring lung surfactant. Other examples include dimyristoylphosphatidycholine (DMPC), dimyristoylphosphatidylglycerol (DMPG), dipalmitoylphosphatidylglycerol (DPPG), distearoylphosphatidylcholine (DSPC), distearoylphosphatidylglycerol (DSPG), dioleylphosphatidylethanolamine (DOPE), dioleoylphosphatidylcholine (DOPC), dimyristoylphosphatidylethanolamine (DMPE), dipalmitoylphosphatidylethanolamine (DPPE), and mixed phospholipids like palmitoylstearoylphosphatidyl-choline (PSPC) and palmitoylstearolphosphatidylglycerol (PSPG), and single acylated phospholipids like mono-oleoyl-phosphatidylethanolamine (MOPE).

The sterols can include, cholesterol, esters of cholesterol including cholesterol hemi-succinate, salts of cholesterol including cholesterol hydrogen sulfate and cholesterol sulfate, ergosterol, esters of ergosterol including ergosterol hemi-succinate, salts of ergosterol including ergosterol hydrogen sulfate and ergosterol sulfate, lanosterol, esters of lanosterol including lanosterol hemi-succinate, salts of lanosterol including lanosterol hydrogen sulfate and lanosterol sulfate.

Other lipids suitable for preparing the lipid particles include sphigomyelin, triglycerides, gangliosides, lysoPC, PEG-lipid, and surfactants.

In one embodiment of the invention the lipid composition contains a phosphatidylethanolamine (PE) such as DMPE, DPPE, or DOPE, and a phosphatidylcholine (PC) such as DMPC, DPPC, or DOPC. The amount of lipid present in the lipid particles can be anywhere from about 1 to about 99 % by weight. In another embodiment the amount of lipid present in the lipid particles can be anywhere from about 2, 5, 10, 15, 20, 25, 30, 35, 40, 45, 50, 55, 60, 65, 70, 75, 80, 85, or 90 to about 99 % by weight. When more than one lipid is present the combined weight percent may be anywhere from about 1 to about 99 % of the lipid particle. When more than one lipid is present the ratio of the lipids may be anywhere from about 1 to about 99 by weight or by moles. In a further embodiment, when two lipids are present in the lipid particles, the ratio by weight or by mole of the lipids may be about 1:1, 1.5:1, 2:1, 2.5:1, 3:1, 3.5:1, 4:1, 4.5:1, 5:1, 10:1, 20:1, 30:1, 40:1, 50:1, 60:1, 70:1, 80:1, or about 90:1. In one embodiment, a PE and a PC lipid are present in the lipid particles wherein the molar ratio by weight of PE to PC is at least about 1. In a further embodiment, the DOPE and DMPC are present in the lipid particle, wherein the molar ratio of DOPE to DMPC is at least about 0.5.

Lipid Particles

The lipid particles disclosed herein have a number of unique properties compared to previously disclosed lipid particles. The hydrophobic bioactive agent complexes with an inverted hexagonal phase-forming lipid at temperatures above the transition temperature (for the lamellar to inverted hexagonal phase transition) of the inverted hexagonal phase forming lipid. Formation of the lipid particles requires the presence of the hydrophobic bioactive agent. The concentration of the lipid(s) is generally more dilute than previously observed. The lipid concentration is generally less than about 8% by weight, and generally about 4, 3, 2, or 1% by weight. Also, preferably, one of the lipids is an inverted hexagonal phase-forming lipid such as a PE. Although an inverted hexagonal phase-forming lipid is used to prepare the lipid particles, the final lipid particle is a solid lacking an inverted hexagonal phase. Table 1 shows the effect the PE transition temperature has on lipid particle formation. Paclitaxel is the hydrophobic bioactive agent.

TABLE 1
The effects of temperature on lipid particle formation*.

| | | Formation of | Transition |
|---|---|---|---|
| | | paclitaxel-PE-PC | temperature of PE** |
| PE | PC | particulate | (° C.) |
| DMPE | DMPC | No | 123 |
| DMPE | DPPC | No | 123 |
| DMPE | DOPC | No | 123 |
| DPPE | DMPC | No | 123 |
| DPPE | DPPC | No | 123 |
| DPPE | DOPC | No | 123 |
| DOPE | DMPC | Yes | 10 |
| DOPE | DPPC | Yes | 10 |
| DOPE | DOPC | Yes | 10 |

*Each formulation contains 15 mg/mL paclitaxel, 15 mg/mL PE, and 10 mg/mL PC. Each formulation was prepared at room temperature.
**Transition temperature for the lamellar to inverted hexagonal phase is from Seddon, J. M., Cevc, G., Marsh, D., Biochemistry, 1983, 22, 1280 for DMPE and DPPE. Data for DMPE was obtained in the presence of 2.4 M NaCl. Data for DOPE is from Cullis, P. R. and de Kruijff, B., Biochim. Biophys. Acta, 1978, 513, 31.

Table 2 shows the importance of formation of complex between the hydrophobic bioactive agent (paclitaxel) and an inverted hexagonal phase-forming lipid (PE) to lipid particle formation. In the absence of hydrophobic drug the lipid particle does not form, indicating that the inverted hexagonal phase itself does not serve as the core of the lipid particle disclosed here.

TABLE 2
Effect of paclitaxel on formation of lipid particle.

| | PE | PC | Formation of |
|---|---|---|---|
| Paclitaxel | (15 mg/mL) | (10 mg/mL) | lipid particle |
| No paclitaxel | DOPE | DMPC | No |
| | | DPPC | No |
| | | DOPC | No |
| Paclitaxel | | DMPC | Yes |
| (15 mg/mL) | | DPPC | Yes |
| | | DOPC | Yes |
| | No PE | DMPC | No |
| | | DPPC | No |
| | | DOPC | No |

The results indicate that the hydrophobic bioactive agent is an essential component of formation of the lipid particles. It is believed that this particular formulation is not an entrapment of paclitaxel in PE-PC delivery vehicle, but a paclitaxel-PE complex fragmented and stabilized in the presence of an amphiphile (PC) by sonication or homogenization.

Table 3 demonstrates that various amphiphiles can be used for stabilizing the lipid particles.

TABLE 3
Effect of other fragmenting stabilizing lipids.

| Hydrophobic | | | Lipid |
|---|---|---|---|
| bioactive | | | particle |
| agent | PE | Fragmenting stabilizer | formation |
| Paclitaxel | Dioleoylphospha- | Didecanoylphosphatidyl- | Yes |
| | tidylethanolamine | choline | |
| | (DOPE) | Dimyristoylphosphatidyl- | Yes |
| | | serine | |
| | | Dipalmitic glycerol | No |
| | | Ganglioside | Yes |
| | | 1-Palmitoyl-2-oleoyl- | Yes |
| | | phosphatidylglycerol | |
| | | Sphingomyelin | Yes |

The lipid particles of the present invention have a hydrophobic bioactive agent to lipid ratio anywhere from about 3.0, 3.5, 4.0, 4.5, 5.0, 5.5, 6.0, 6.5, 7.0, 7.5, 8.0, 8.5, or 9.0:10, which corresponds to about 30, 35, 40, 45, 50, 55, 60, 65, 70, 75, 80, or 85% to about 90% of hydrophobic agent to total lipid particle by weight. In another embodiment, the hydrophobic bioactive agent to lipid ratio is about 1:0.7 to about 1:2.5 by weight, or about 30% to about 60% of hydrophobic bioactive agent to total lipid particle by weight. In another embodiment, the hydrophobic bioactive agent to lipid ratio is anywhere from about 1:1.5 to about 1:2.0 by weight, or about 33% to about 40% of hydrophobic bioactive agent to total lipid particle by weight. In another embodiment, the hydrophobic bioactive agent to lipid ratio is about 1:0.7 by weight, or about 60% of hydrophobic bioactive agent to total lipid particle by weight. Particle size as measured by mean diameter of the lipid particles of the present invention is anywhere from about 200 to about 1000 nm. In another embodiment, the particle size is anywhere from about 400 to about 700 nm. In another embodiment, the particle is about 500 to 600 nm.

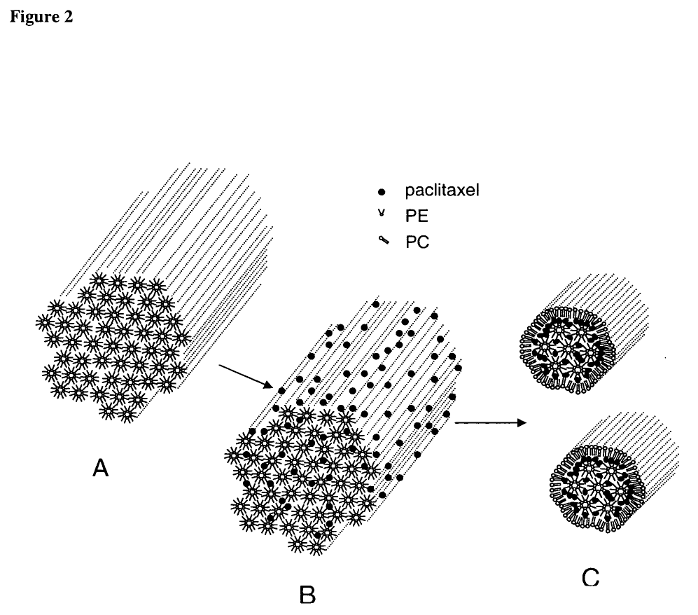

FIG. 2 depicts the structure of the bioactive containing lipid particles of the present invention. FIG. 2A is the reverse hexagonal(II) phase of the lipid. Because the hydrophobic hydrocarbon region is exposed to aqueous environment, the structure grows quite large (can be a few mm). The structure usually breaks down as big chunks so that entropy effects can overcome the thermodynamically unfavorable hydrophobic hydrocarbon-water contact by physical agitation.

Paclitaxel is oil-soluble (e.g. BMS's Taxol uses castrol oil to dissolve paclitaxel). FIG. 2B shows paclitaxel dissolved in the hydrocarbon region (oily part of lipids). Here sonication (or other shear force) is required to disrupt the structure momentarily to get paclitaxel to interact with the hidden hydrophobic regions of the lipid chunks (still, large chucks remain).

The structure in FIG. 2B still has a huge hydrophobic surface exposed to an aqueous environment. Again to overcome this thermodynamically unfavorable situation, the structure remains as big chunks. This structure can be broken down to a smaller size by sonication and stabilized (kept small) by an amphiphile coating monolayer. Of course, hydrocarbon is covering the surface of the structure in FIG. 2B and the hydrophilic head is exposed to water, providing a thermodynamically favorable structure. This allows smaller structures to be stable. (FIG. 2)C).

This sizing & stabilizing process requires the presence of the hydrophobic bioactive agent, indicating that incorporation of the hydrophobic bioactive agent in the structure in FIG. 2A (PE in inverted hexagonal phase) leads to the PE-hydrophobic bioactive agent complex in FIG. 2B.

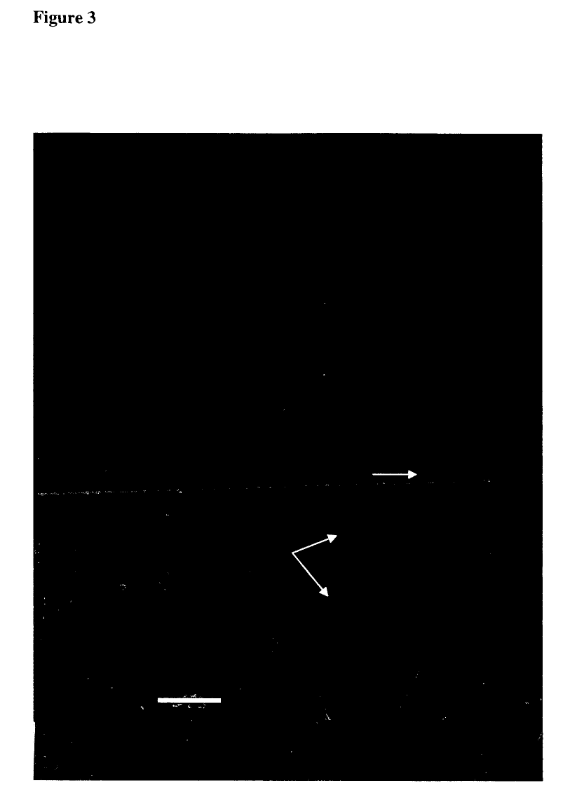

FIG. 3 depicts the freeze-fracture electron microscope (EM) image of the lipid particles of the present invention where the lipid is DOPE, the hydrophobic bioactive agent is paclitaxel, and the amphiphile is DMPC. The image was taken before size separation by centrifugation. Larger particles are dominantly observed because larger objects are more readily sampled for freeze-fracture EM images. Arrows indicate particles with the sizes determined from the final product. The white bar represents 1 micron.

Methods of Preparing the Lipid Particles

In one embodiment, the hydrophobic bioactive agent (e.g. paclitaxel) and an inverted hexagonal phase-forming lipid (e.g. DOPE) are mixed in an aqueous solution by a shear-force generating method such as homogenization, sonication, grinding, milling, or atomization. An amphiphile (e.g. DMPC) is added to the mixture and then further mixed by a shear-force generating method such as homogenization, sonication, grinding, milling, atomization, until a milky suspension (lipid particles) forms. The resulting lipid particles may then be fractionated to obtain particles with a certain size distribution or to remove the larger lipid particles. The fractionation method includes centrifugation, density gradient centrifugation, gravitational settlement, filtration, or a gel-permeation chromatographic method.

In another embodiment, the hydrophobic bioactive agent (e.g. paclitaxel) and the inverted hexagonal phase-forming lipid (e.g. DOPE) are codissolved in a non-aqueous solvent (e.g. ethanol) and infused in an aqueous solution, followed by removal of the non-aqueous solvent using evaporation, dialysis, or diafiltration. An amphiphile (e.g. DMPC) is dissolved in a non-aqueous solvent (e.g. ethanol) and infused in an aqueous solution, followed by a removal of the non-aqueous solvent using evaporation, dialysis, or diafiltration. These two suspensions prepared separately are mixed together by a shear-force generating method such as homogenization, sonication, grinding, milling, atomization, until the milky suspension (lipid particles) forms. The resulting lipid particles may then be fractionated to obtain particles with a certain size distribution or to remove larger lipid particles. The fractionation method includes centrifugation, density gradient centrifugation, gravitational settlement, filtration, or a gel-permeation chromatographic method.

The above methods may be carried out aseptically by sterile filtering the individual solutions prior to either solvent removal or combining the solutions.

In another embodiment, the lipid particle prepared as above may be freeze-dried in the presence of cryoprotactant such as lactose for an extended shelf life. The lipid particles are reconstituted by resuspending the freeze-dried lipid particles into an aqueous solution.

Inhalation Devices

The lipid particles comprising a bioactive agent may be delivered in a variety of ways known in the art. One method of delivery particularly suitable for the treatment of lung diseases is by inhalation. The inhalation delivery device can be a nebulizer, a metered dose inhaler (MDI) or a dry powder inhaler (DPI). The device can contain and be used to deliver a single dose of the lipid compositions or the device can contain and be used to deliver multi-doses of the lipid compositions of the present invention. In another embodiment, the nebulizer is envisioned to be disposable.

A nebulizer type inhalation delivery device can contain the compositions of the present invention as a solution, usually aqueous, or a suspension. In generating the nebulized spray of the compositions for inhalation, the nebulizer type delivery device may be driven ultrasonically, by compressed air, by other gases, electronically or mechanically (including, for example, a vibrating porous membrane). The ultrasonic nebulizer device usually works by imposing a rapidly oscillating waveform onto the liquid film of the formulation via an electrochemical vibrating surface. At a given amplitude the waveform becomes unstable, whereby it disintegrates the liquids film, and it produces small droplets of the formulation. The nebulizer device driven by air or other gases operates on the basis that a high pressure gas stream produces a local pressure drop that draws the liquid formulation into the stream of gases via capillary action. This fine liquid stream is then disintegrated by shear forces. The nebulizer may be portable and hand held in design, and may be equipped with a self contained electrical unit. The nebulizer device can consist of a nozzle that has two coincident outlet channels of defined aperture size through which the liquid formulation can be accelerated. This results in impaction of the two streams and atomization of the formulation. The nebulizer may use a mechanical actuator to force the liquid formulation through a multiorifice nozzle of defined aperture size(s) to produce an aerosol of the formulation for inhalation. In the design of single dose nebulizers, blister packs containing single doses of the formulation may be employed.

In the present invention the nebulizer is employed to ensure the sizing of aqueous droplets containing the drug-lipid particles is optimal for positioning of the particle within, for example, the lungs. Typical droplet sizes for the nebulized lipid composition are from about I to about 5 microns.

For use with the nebulizer, the lipid composition preferably contains an aqueous component. Typically there is at least about 80% by weight and preferably, at least about 90% by weight of the aqueous component in the lipid composition to be administered with a nebulizer. The aqueous component may include for example, saline. In addition, the aqueous component may include up to about 20% by weight of an aqueous compatible solvent such as ethanol.

Total administration time using a nebulizer will depend on the flow rate and the concentration of the bioactive agent in the lipid composition. Variation of the total administration time is within the purview of those of ordinary skill in the art. Generally, the flow rate of the nebulizer will be at least about 0.15 mL/min, for example, a flow rate of about 0.2 mL/min is typical. By way of example, administration of a dose of about 24 mg/m² of a bioactive agent using a lipid composition having a concentration of about 1 mg/mL of bioactive agent would be about 4 hours (assuming a patient's body surface area is about 2 m²). This administration time may, for example, be split into two administration sessions given over the course of one or two days to complete one treatment cycle.

In alternative embodiments, a metered dose inhalator (MDI) can be employed as the inhalation delivery device of the inhalation system. This device is pressurized (pMDI) and its basic structure consists of a metering valve, an actuator and a container. A propellant is used to discharge the formulation from the device. The composition can consist of particles of a defined size suspended in the pressurized propellant(s) liquid, or the composition can be in a solution or suspension of pressurized liquid propellant(s). The propellants used are primarily atmospheric friendly hydroflourocarbons (HFCs) such as 134a and 227. Traditional chloroflourocarbons like CFC-1 1, 12 and 114 are used only when essential. The device of the inhalation system may deliver a single dose via, e.g., a blister pack, or it may be multi dose in design. The pressurized metered dose inhalator of the inhalation system can be breath actuated to deliver an accurate dose of the lipid based formulation. To insure accuracy of dosing, the delivery of the formulation may be programmed via a microprocessor to occur at a certain point in the inhalation cycle. The MDI may be portable and hand held.

In another alternative embodiment, a dry powder inhalator (DPI) can be used as the inhalation delivery device of the inhalation system. This device's basic design consists of a metering system, a powdered composition and a method to disperse the composition. Forces like rotation and vibration can be used to disperse the composition. The metering and dispersion systems may be mechanically or electrically driven and may be microprocessor programmable. The device may be portable and hand held. The inhalator may be multi or single dose in design and use such options as hard gelatin capsules, and blister packages for accurate unit doses. The composition can be dispersed from the device by passive inhalation; i.e., the patient's own inspiratory effort, or an active dispersion system may be employed. The dry powder of the composition can be sized via processes such as jet milling, spray dying and supercritical fluid manufacture. Acceptable excipients such as the sugars mannitol and maltose may be used in the preparation of the powdered formulations. These are particularly important in the preparation of freeze dried liposomes and lipid complexes. These sugars help in maintaining the liposome's physical characteristics during freeze drying and minimizing their aggregation when they are administered by inhalation. The hydroxyl groups of the sugar may help the vesicles maintain their tertiary hydrated state and help minimize particle aggregation.

The inventive method is particularly well-suited for the pre-treatment and treatment of lung diseases such as lung cancer. In addition, both primary and metastatic lung cancers are excellent candidates for the method of the invention.

Dosages

Administration of the compositions of the present invention will be in an amount sufficient to achieve a therapeutic effect as recognized by one of ordinary skill in the art.

The dosage of any compositions of the present invention will vary depending on the symptoms, age and body weight of the patient, the nature and severity of the disorder to be treated or prevented, the route of administration, and the form of the subject composition. Any of the subject formulations may be administered in a single dose or in divided doses. Dosages for the compositions of the present invention may be readily determined by techniques known to those of skill in the art or as taught herein.

In certain embodiments, the dosage of the subject compounds will generally be in the range of about 0.01 ng to about 10 g per kg body weight, specifically in the range of about 1 ng to about 0.1 g per kg, and more specifically in the range of about 100 ng to about 10 mg per kg.

An effective dose or amount, and any possible affects on the timing of administration of the formulation, may need to be identified for any particular composition of the present invention. This may be accomplished by routine experiment as described herein, using one or more groups of animals (preferably at least 5 animals per group), or in human trials if appropriate. The effectiveness of any subject composition and method of treatment or prevention may be assessed by administering the composition and assessing the effect of the administration by measuring one or more applicable indices, and comparing the post-treatment values of these indices to the values of the same indices prior to treatment.

The precise time of administration and amount of any particular subject composition that will yield the most effective treatment in a given patient will depend upon the activity, pharmacokinetics, and bioavailability of a subject composition, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing.

While the subject is being treated, the health of the patient may be monitored by measuring one or more of the relevant indices at predetermined times during the treatment period. Treatment, including composition, amounts, times of administration and formulation, may be optimized according to the results of such monitoring. The patient may be periodically reevaluated to determine the extent of improvement by measuring the same parameters. Adjustments to the amount(s) of subject composition administered and possibly to the time of administration may be made based on these reevaluations.

Treatment may be initiated with smaller dosages which are less than the optimum dose of the compound. Thereafter, the dosage may be increased by small increments until the optimum therapeutic effect is attained.

The use of the subject compositions may reduce the required dosage for any individual agent contained in the compositions (e.g., the steroidal anti inflammatory drug) because the onset and duration of effect of the different agents may be complimentary.

Toxicity and therapeutic efficacy of subject compositions may be determined by standard pharmaceutical procedures in cell cultures or experimental animals, e.g., for determining the LD₅₀ and the ED₅₀.

The data obtained from the cell culture assays and animal studies may be used in formulating a range of dosage for use in humans. The dosage of any subject composition lies preferably within a range of circulating concentrations that include the ED₅₀ with little or no toxicity. The dosage may vary within this range depending upon the dosage form employed and the route of administration utilized. For compositions of the present invention, the therapeutically effective dose may be estimated initially from cell culture assays.

In general, the doses of an active agent will be chosen by a physician based on the age, physical condition, weight and other factors known in the medical arts.

Formulation

The lipid particles presently disclosed may be administered by various means, depending on their intended use, as is well known in the art. For example, if compositions of the present invention are to be administered orally, they may be formulated as tablets, capsules, granules, powders or syrups. Alternatively, formulations of the present invention may be administered parenterally as injections (intravenous (IV), intramuscular or subcutaneous), drop infusion preparations or suppositories. For application by the ophthalmic mucous membrane route, compositions of the present invention may be formulated as eyedrops or eye ointments. These formulations may be prepared by conventional means, and, if desired, the compositions may be mixed with any conventional additive, such as an excipient, a binder, a disintegrating agent, a lubricant, a corrigent, a solubilizing agent, a suspension aid, an emulsifying agent or a coating agent.

In formulations of the subject invention, wetting agents, emulsifiers and lubricants, such as sodium lauryl sulfate and magnesium stearate, as well as coloring agents, release agents, coating agents, sweetening, flavoring and perfuming agents, preservatives and antioxidants may be present in the formulated agents.

Subject compositions may be suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of composition that may be combined with a carrier material to produce a single dose vary depending upon the subject being treated, and the particular mode of administration.

Methods of preparing these formulations include the step of bringing into association compositions of the present invention with the carrier and, optionally, one or more accessory ingredients. In general, the formulations are prepared by uniformly and intimately bringing into association agents with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), each containing a predetermined amount of a subject composition thereof as an active ingredient. Compositions of the present invention may also be administered as a bolus, electuary, or paste.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Liquid dosage forms for oral administration include pharmaceutically acceptable emulsions, microemulsions, solutions, suspensions, syrups and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

Suspensions, in addition to the subject composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable non-irritating excipients or carriers comprising, for example, cocoa butter, polyethylene glycol, a suppository wax or a salicylate, and which is solid at room temperature, but liquid at body temperature and, therefore, will melt in the body cavity and release the active agent. Formulations which are suitable for vaginal administration also include pessaries, tampons, creams, gels, pastes, foams or spray formulations containing such carriers as are known in the art to be appropriate.

Dosage forms for transdermal administration of a subject composition includes powders, sprays, ointments, pastes, creams, lotions, gels, solutions, patches and inhalants. The active component may be mixed under sterile conditions with a pharmaceutically acceptable carrier, and with any preservatives, buffers, or propellants which may be required.

The ointments, pastes, creams and gels may contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a subject composition, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

As discussed previously, compositions and compounds of the present invention may alternatively be administered by aerosol. A non-aqueous (e.g., fluorocarbon propellant) suspension could be used. Sonic nebulizers may be used because they minimize exposing the agent to shear, which may result in degradation of the compounds contained in the subject compositions.

Ordinarily, an aqueous aerosol is made by formulating an aqueous solution or suspension of a subject composition together with conventional pharmaceutically acceptable carriers and stabilizers. The carriers and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Pharmaceutical compositions of this invention suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity may be maintained, for example, by the use of coating materials, such as lecithin, by the maintenance of the required particle size in the case of dispersions, and by the use of surfactants.

The lipid particles can be formulated for parenteral administration, as for example, for subcutaneous, intramuscular, intratracheal, intraperitoneal, intratumor, or intravenous injection, e.g., the lipid particles can be provided in a sterile solution or suspension (collectively hereinafter “injectable solution”). The injectable solution is formulated such that the amount of hydrophobic bioactive agent (or agents) provided in a 200 cc bolus injection would provide a dose of at least the median effective dose, or less than 100 times the ED₅₀, or less than 10 or 5 times the ED₅₀. The injectable solution may be formulated such that the total amount of hydrophobic agent (or agents) provided in 100, 50, 25, 10, 5, 2.5, or 1 cc injections would provide an ED₅₀ dose to a patient, or less than 100 times the ED₅₀, or less than 10 or 5 times the ED₅₀. In other embodiments, the amount of hydrophobic bioactive agent (or agents) provided in a total volume of 100 cc, 50, 25, 5 or 2 cc to be injected at least twice in a 24 hour time period would provide a dosage regimen providing, on average, a mean plasma level of the hydrophobic bioactive agent(s) of at least the ED₅₀ concentration, or less than 100 times the ED₅₀, or less than 10 or 5 times the ED₅₀. In other embodiments, a single dose injection provides about 0.25 mg to 1250 mg of hydrophobic bioactive agent.

Efficacy of Treatment

The efficacy of treatment with the subject compositions may be determined in a number of fashions known to those of skill in the art.

In one exemplary method, when treatment is for lung cancer, the median rate of decrease in tumor or lesion size from treatment with a subject composition may be compared to other forms of treatment with the particular therapeutic agent contained in the subject composition, or with other therapeutic agents. The decrease in tumor or lesion size for treatment with a subject composition as compared to treatment with another method may be 10, 25, 50, 75, 100, 150, 200, 300, 400% greater or even more. The period of time for observing any such decrease may be about 1, 3, 5, 10, 15, 30, 60 or 90 or more hours. The comparison may be made against treatment with the particular therapeutic agent contained in the subject composition, or with other therapeutic agents, or administration of the same or different agents by a different method, or administration as part of a different drug delivery device than a subject composition. The comparison may be made against the same or a different effective dosage of the various agents.

Alternatively, a comparison of the different treatment regimens described above may be based on the effectiveness of the treatment, using standard indices known to those of skill in the art. One method of treatment may be 10%, 20%, 30%, 50%, 75%, 100%, 150%, 200%, 300% more effective, than another method.

Alternatively, the different treatment regimens may be analyzed by comparing the therapeutic index for each of them, with treatment with a subject composition as compared to another regimen having a therapeutic index two, three, five or seven times that of, or even one, two, three or more orders of magnitude greater than, treatment with another method using the same or different therapeutic agents.

Kits This invention also provides kits for conveniently and effectively implementing the methods of this invention. Such kits comprise any subject composition, and a means for facilitating compliance with methods of this invention. Such kits provide a convenient and effective means for assuring that the subject to be treated takes the appropriate active in the correct dosage in the correct manner. The compliance means of such kits includes any means which facilitates administering the actives according to a method of this invention. Such compliance means include instructions, packaging, and dispensing means, and combinations thereof. Kit components may be packaged for either manual or partially or wholly automated practice of the foregoing methods. In other embodiments involving kits, this invention contemplates a kit including compositions of the present invention, and optionally instructions for their use.

### EXEMPLIFICATION

### Example 1

Formation of lipid particles comprising paclitaxel (a). Paclitaxel was suspended in deionized water. DOPE was added to the paclitaxel suspension. The DOPE and paclitaxel were mixed by brief sonication to form larger complex precipitates. DMPC was added to paclitaxel-PE complex. The mixture was again mixed by sonication until it formed a milky suspension.

The resulting particles were mostly uniform but still comprised a few large particles. To remove the larger particles the sample was centrifuged (low speed). The top suspension was collected as a final formulation and analyzed for paclitaxel and lipid levels. The results are presented in Table 4.

TABLE 4
Lipid and paclitaxel levels.

| | Paclitaxel | Total Lipid | Lipid/Drug |
|---|---|---|---|
| Initial Charge | 15.0 mg/mL | 25.0 mg/mL | 1.7 |
| After Process | 10.4 mg/mL | 16.5 mg/mL | 1.6 |
| Recovery | 69.3% | 66.0% | 94% |

Table 5 shows the effect of nebulization on the lipid particles.

TABLE 5
Effects of nebulization.

| | | | | Particle | Cytotox- |
|---|---|---|---|---|---|
| | Paclitaxel | Lipid | Lipid/ | Size | icity*, |
| | (mg/mL) | (mg/mL) | Drug | (solid) | ID₅₀ |
| Lipid | 10.5 | 16.5 | 1.6 | 0.50 | 43 ng/mL |
| Particle | | | | (inten- | |
| | | | | sity-wt) | |
| Nebulyzate | 12.2 | 18.0 | 1.5 | 0.45 | 38 ng/mL |
| | | | | (inten- | |
| | | | | sity-wt) | |

*Cytotoxicity was measured by MTT assay. The cell line used was H460 Human lung carcinoma (non-small cell lung carcinoma). ID₅₀ is the dose (concentration) of the drug that causes 50% cell growth inhibition. ID₅₀ is 94 ng/mL for free paclitaxel.

### Example 2

Formation of lipid particles comprising paclitaxel (b). Paclitaxel was suspended in deionized water. DOPE was added to the paclitaxel suspension. The DOPE and hydrophobic paclitaxel were mixed by brief sonication to form large complex precipitates. DMPC was added to the paclitaxel-PE complex. The mixture was again sonicated until it reached a milky suspension.

After the process the resulting particles were mostly uniform, but there were still a few large particles. To remove larger particles the sample was centrifuged (low speed). The top suspension (90 % volume) was collected and centrifuged (high speed) again. The supernatant was discarded to remove potentially small vesicles and the pellet was reconstituted with distilled water. The pellet was analyzed for paclitaxel and lipid levels. The results are presented in Table 6.

TABLE 6
Lipid and paclitaxel levels.

| | Paclitaxel | DOPE | DMPC |
|---|---|---|---|
| Initial | 15.0 mg/mL | 15.0 mg/mL | 10 mg/mL |
| Charge | | | |
| After | 5.8 mg/mL | 2.8 mg/mL | 1.2 mg/mL |
| Process | (90%) | (90%) | (90%) |
| Recovery | 35% | 17% | 11% |

Drug/lipid ratio by weight is 4.8/2.3/1 (paclitaxel/dioleoylphosphatidylethanolamine/dimyristoylphosphatidylcholine).

Table 7 summarizes the mean diameter of the lipid particles.

TABLE 7
Narrow particle size distribution range.

| | Intensity-weighted | Volume-weighted | Number-weighted |
|---|---|---|---|
| Mean | 375.3 nm | 403.6 nm | 308.6 nm |
| Diameter* | | | |

*Chi squared was 0.808 (Gaussian distribution).

### Example 3

Formation of lipid particles comprising various bioactive agents. The initial composition for each formulation was 15 mg/mL of bioactive agent, 15 mg/mL of DOPE, and 10 mg/mL of DMPC. An aqueous mixture of bioactive agent and lipid mixture was sonicated until the mixture became a suspension. The suspension was centrifuged to settle large particles and the top 90% of the suspension was collected and analyzed. The results are shown in Table 8.

TABLE 8
Lipid particles comprising various bioactive agents.

| | Bio- | | | | | |
|---|---|---|---|---|---|---|
| | active | DOPE | DMPC | Drug/ | Bioactive | Mean |
| Bioactive | agent | (mg/ | (mg/ | Lipid | agent | particle |
| agent | (mg/mL) | mL) | mL) | (w/w) | recovery | size |
| Ampho- | 4.3 | 9 | 9 | 1/4.2 | 25.8% | 436 nm |
| tericin B | | | | | | |
| Campto- | 11.9 | 9 | 10 | 1/1.6 | 71.4% | 626 nm |
| thecin | | | | | | |
| Cisplatin | 8.2 | 10 | 8 | 1/2.2 | 49.2% | 520 nm |

The above results demonstrate that the lipid particles can be formed not only with paclitaxel but also other hydrophobic bioactive agents or bioactive agents that form crystals in aqueous solution. The characteristics of these formulations vary with different bioactive agents. They all, however, show excellent drug recovery and high drug to lipid ratios.

### Example 4

Effect of paclitaxel-PE-PC particulates on cytotoxicity of paclitaxel : Enhancement of cytotoxicity of paclitaxel by the lipid complex formulation. Cytotoxicity was measured by MTT assay. The cell line used was H460 Human lung carcinoma (non-small cell lung carcinoma). Enhancement was measured as relative cytotoxicity defined as (ID₅₀ of the formulation)/(ID₅₀ of free paclitaxel). ID₅₀ being the dose (concentration) of the drug that causes 50% cell growth inhibition. The paclitaxel-PE-PC particulate formulation doubled the cytotoxicity of paclitaxel as shown in Table 9. This believed to be due to the better membrane permeability of the lipid complex formulation than free paclitaxel, causing higher cytoplasmic concentration of the drug.

TABLE 9
Relative cytotoxicity of paclitaxel associated with
lipid particle compared to free paclitaxel.

| Lot # | Relative Cytotoxicity |
|---|---|
| 1 | 2.2 |
| 2 | 1.9 |

### Example 5

Aseptic process of making paclitaxel-PE-PC complex. Paclitaxel and DOPE were dissolved in ethanol and sterile-filtered before addition to sterile water. The mixture was dialyzed under sterile conditions. Separately, DMPC dissolved in ethanol was also sterile-filtered and added into sterile water. This mixture was dialyzed under sterile condition. The dialyzation process can be replaced by diafiltration or evaporation methods to remove the organic solvent. The mixture was then sonicated until a milky suspension was formed. The suspension was centrifuged and the top 90% of total volume was collected. The results are shown in Table 10.

TABLE 10
Lipid particles comprising paclitaxel.

| | Paclitaxel | DOPE | DMPC | Drug/lipid |
|---|---|---|---|---|
| Initial charge | 10 mg/ml | 10 mg/ml | 15 mg/ml | 1/2.5 |
| After process | 4 mg/ml | 3.4 mg/ml | 6.3 mg/ml | 1/2.4 |
| Recovery | 36% | 30.6% | 37.8% | 96% |

### Example 6

Effect of freeze-drying (lyophilization) on paclitaxel-PE-PC particles. The paclitaxel-PE-PC particles were prepared as in Example 2. Before freeze-drying, 5% wt/vol lactose was added to the formulation as a cryoprotactant. After freeze drying, the formulation was reconstituted and the original paclitaxel-PE-PC particles were recovered unchanged as shown in Table 11.

TABLE 11
Effect of freeze-drying on paclitaxel-PE-PC particles.

| | | | Drug/ | Mean diameter |
|---|---|---|---|---|
| | Paclitaxel | Total lipid | lipid | of lipid Particle |
| Before Freeze- | 10.4 mg/ml | 16.5 mg/ml | 1/1.6 | 0.52 μm |
| drying | | | | |
| Reconstituted | 9.4 mg/ml | 15.4 mg/ml | 1/1.6 | 0.54 μm |
| after freeze- | | | | |
| drying | | | | |

These results demonstrate that the formulations disclosed herein can be freeze-dried to obtain superior shelf-life.

### Example 7

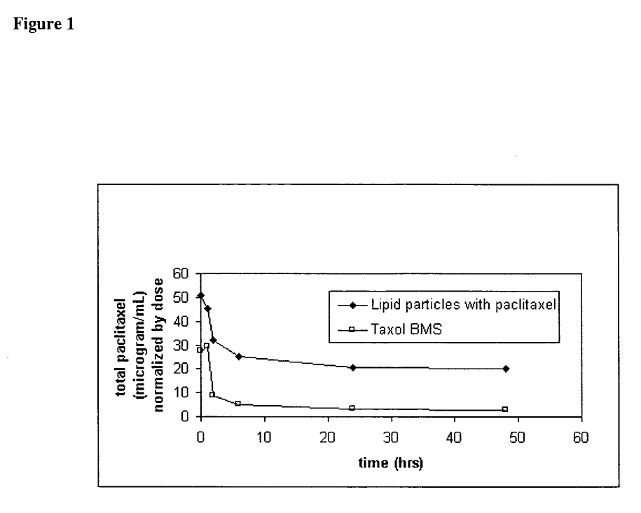

In vivo pharmacokinetic study of lipid particles with paclitaxel vs. taxol (micellar formulation, BMS) via intratracheal instillation in Sprague/Dawley rats. The major clearance of paclitaxel in rat lung occurs during first 6 hours after IT instillation for both formulations (FIG. 1). It would be impossible to make an accurate estimate for drug level for time zero because the pulmonary clearance is immediate and fast, especially for free drug or smaller particles such as micelles. Even immediate sacrifice of the animal after treatment (time zero) resulted in substantially lower drug level for taxol. For the lipid particles with paclitaxel, about 40% of paclitaxel level at time zero was maintained after 6 hours through 48 hours (the end point of the study). On the other hand, most paclitaxel was cleared after 6 hours for taxol. This demonstrates the pulmonary depot effect of the lipid particles with paclitaxel while showing no such an effect for taxol (a micellar formulation of paclitaxel). Furthermore, this indicates that the newly formulated lipid particles with paclitaxel stays in the lung much longer than taxol, proposing a better therapeutic strategy for cancer treatment.

### Example 8

Lipid particles comprising paclitaxel are stable during long-term storage as well as during nebulization. A major stability problem for formulations comprising hydrophobic drugs such as paclitaxel is that the drug being crystallizes out to the aqueous solution, resulting in the formation of aggregates. This potential crystallization was monitored by particle size measurement. After 2 years of storage at 4° C. the particle size remained same, showing no sign of crystallization. The particle size remained the same even during nebulization using a high shear force as shown in Table 12.

TABLE 12
Particle size (mean diameter) of the lipid particles with paclitaxel
measured by the Quasi-Elastic Light Scattering method (QELS).

| | Intensity- | Volume- | Number- |
|---|---|---|---|
| | weighted | weighted | weighted |
| At time zero | 0.50 μm | 0.62 μm | 0.22 μm |
| After nebulization* | 0.46 μm | 0.53 μm | 0.28 μm |
| After 2 years of storage at 4° C. | 0.47 μm | 0.55 μm | 0.27 μm |

*The nebulizate was collected for 20 min. by a cold impinger connected to the mouth piece of a Pari LC Star jet nebulizer.

### Example 9

PC coating of the lipid particles is a monolayer. The ratio of probe lipids on the surface and within the lipid complex was determined and compared for liposomes and the lipid particles of the present invention. DMPC liposomes were prepared with 0.5 wt % fluorescence probe (NBD: N-7-nitro-2,1,3-benzoxadiazol-4-yl) lipid and sonicated by a bath sonicator for 10 min. The probe lipids evenly distribute to both inside and outside of the bilayer. Addition of a membrane-impermeable reducing agent, dithionite, quenches the fluorescence of the probe lipid located on only the surface of the liposomes. McIntyre, J. G. & Sleight, R. G. (1991) Biochemistry 30, 11819-11827. The ratio between the probes located on the surface and inside liposomes was estimated: % probe lipid on the surface=(Initial fluorescence intensity−Fluorescence intensity after quenching)×100/initial fluorescence intensity.

Separately, DMPC liposomes with 2 wt % NBD lipids were added in a DOPE/paclitaxel mixture to produce the lipid particles. To exclude residual liposomes containing probes, the sample was centrifuged at high speed after sonication. The supernatant containing most of the liposomes was removed. The remaining pellet was resuspended with distilled water and then centrifuged at low speed to settle large particles. The supernatant was collected and used for the lipid particles with paclitaxel. Table 13 lists and compares the ratios for the two types of lipid complexes.

TABLE 13
The ratio between the probes located on the surface
and inside the liposomes and lipid particles.

| | % probe lipid located on the surface |
|---|---|
| | of the liposomes or lipid particles |
| DMPC liposomes | 46 |
| Lipid particles | 98 |
| with paclitaxel | |

For liposomes, nearly a half of the probe lipid was located outside of the liposomes, reflecting the structure of bilayer. Conversely, the lipid particles had most of the probe lipids on their surface, reflecting the structure of monolayer.

### INCORPORATION BY REFERENCE

All of the patents and publications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A non-liposomal lipid particle comprising an amphiphile-coated complex of a hydrophobic bioactive agent and an inverted hexagonal phase forming lipid.

2. The lipid particle of claim 1, wherein the bioactive agent is a taxane.

3. The lipid particle of claim 1, wherein the bioactive agent is a platinum complex.

4. The lipid particle of claim 1, wherein the bioactive agent is cisplatin, carboplatin, oxaliplatin, paclitaxel, camptothecin, or topotecin.

5. The lipid particle of claim 1, wherein the bioactive agent is paclitaxel.

6. The lipid particle of claim 1, wherein the bioactive agent is camptothecin.

7. The lipid particle of claim 1, wherein the bioactive agent is cisplatin.

8. The lipid particle of claim 1, wherein the bioactive agent is amphotericin B.

9. The lipid particle of claim 1, wherein the inverted hexagonal phase forming lipid is a phosphatidylethanolamine (PE).

10. The lipid particle of claim 1, wherein the inverted hexagonal phase forming lipid is dioleoylphosphatidylethanolamine (DOPE).

11. The lipid particle of claim 1, the inverted hexagonal phase forming lipid is dimyristoylphosphatidylethanolamine (DMPE).

12. The lipid particle of claim 1, the inverted hexagonal phase forming lipid is dipalmitoylphosphatidylethanolamine (DPPE).

13. The lipid particle of claim 1, wherein the amphiphile is a phosphatidylcholine (PC), phosphatidylglycerol (PG), phosphatidylserine (PS), phosphatidylethanolamine (PE), phosphatidylinositol (PI), phosphatidic acid (PA), sphigomyelin, ganglioside, lysoPC, PEG-lipid, surfactant, or a combination thereof.

14. The lipid particle of claim 1, wherein the amphiphile is dimyristoylphosphatidylcholine (DMPC).

15. The lipid particle of claim 1, wherein the amphiphile is dipalmitoylphosphatidylcholine (DPPC).

16. The lipid particle of claim 1, wherein the amphiphile is dioleoylphosphatidylcholine (DOPC).

17. The lipid particle of claim 1, wherein the amphiphile is didecanoylphosphatidylcholine (DDPC).

18. The lipid particle of claim 1, wherein the amphiphile is dimyristoylphosphatidylserine (DMPS).

19. The lipid particle of claim 1, wherein the amphiphile is brain ganglioside.

20. The lipid particle of claim 1, wherein the amphiphile is 1-palmitoyl-2-oleoylphosphatidylglycerol (POPG).

21. The lipid particle of claim 1, wherein the amphiphile is sphingomyeline.

22. The lipid particle of claim 1, wherein the inverted hexagonal phase forming lipid is DOPE and the amphiphile is DMPC.

23. The lipid particle of claim 1, wherein the inverted hexagonal phase forming lipid is DOPE and the amphiphile is DPPC.

24. The lipid particle of claim 1, wherein the inverted hexagonal phase forming lipid is DOPE and the amphiphile is DOPC.

25. The lipid particle of claim 1, wherein the inverted hexagonal phase forming lipid is DOPE and the amphiphile is DDPC.

26. The lipid particle of claim 1, wherein the inverted hexagonal phase forming lipid is DOPE and the amphiphile is DMPS.

27. The lipid particle of claim 1, wherein the inverted hexagonal phase forming lipid is DOPE and the amphiphile is brain ganglioside.

28. The lipid particle of claim 1, wherein the inverted hexagonal phase forming lipid is DOPE and the amphiphile is POPG.

29. The lipid particle of claim 1, wherein the inverted hexagonal phase forming lipid is DOPE and the amphiphile is sphingomyelin.

30. The lipid particle of claim 1, wherein the inverted hexagonal phase forming lipid is DOPE, the bioactive agent is paclitaxel, and the amphiphile is DMPC.

31. The lipid particle of claim 1, wherein the inverted hexagonal phase forming lipid is DOPE, the bioactive agent is paclitaxel, and the amphiphile is DPPC.

32. The lipid particle of claim 1, wherein the inverted hexagonal phase forming lipid is DOPE, the bioactive agent is paclitaxel, and the amphiphile is DOPC.

33. The lipid particle of claim 1, wherein the inverted hexagonal phase forming lipid is DOPE and the amphiphile is DDPC.

34. The lipid particle of claim 1, wherein the inverted hexagonal phase forming lipid is DOPE, the bioactive agent is paclitaxel, and the amphiphile is DMPS.

35. The lipid particle of claim 1, wherein the inverted hexagonal phase forming lipid is DOPE, the bioactive agent is paclitaxel, and the amphiphile is brain ganglioside.

36. The lipid particle of claim 1, wherein the inverted hexagonal phase forming lipid is DOPE, the bioactive agent is paclitaxel, and the amphiphile is POPG.

37. The lipid particle of claim 1, wherein the inverted hexagonal phase forming lipid is DOPE, the bioactive agent is paclitaxel, and the amphiphile is sphingomyelin.

38. The lipid particle of claim 1, wherein the inverted hexagonal phase forming lipid is DOPE, the bioactive agent is amphotericin B, and the amphiphile is DMPC.

39. The lipid particle of claim 1, wherein the inverted hexagonal phase forming lipid is DOPE, the bioactive agent is camptothecin, and the amphiphile is DMPC.

40. The lipid particle of claim 1, wherein the inverted hexagonal phase forming lipid is DOPE, the bioactive agent is cisplatin, and the amphiphile is DMPC.

41. The lipid particle of claim 1, wherein the cytotoxicity of the bioactive agent as measured by MTT assay using H460 Human lung carcinoma cell line is at least twice the cytotoxicity of the free bioactive agent.

42. The lipid particle of claim 41, wherein the bioactive agent is a platinum complex.

43. The lipid particle of claim 41, wherein the bioactive agent is paclitaxel.

44. A method of preparing the lipid particle of claim 1 comprising:
a) combining a hydrophobic bioactive agent and an inverted hexagonal phase-forming lipid in an aqueous solution;
b) mixing the suspension from step a) by a shear-force generating method;
c) adding an amphiphile to the mixture from step b); and
d) mixing the suspension from step c) by a shear-force generating method at least until a milky suspension forms.

45. The method of claim 44, wherein the suspension from step d) is further fractionated using centrifugation, density gradient centrifugation, or gravitational settlement to obtain particles with a certain size distribution or to remove larger lipid particles.

46. The method of claim 44, wherein the suspension from step d) is further filtered to remove larger lipid particles.

47. The method of claim 44, wherein the suspension from step d) is further fractionated by gel-permeation chromatographic methods to obtain particles with a certain size distribution, or to remove larger lipid particles.

48. The method of claim 44, wherein the shear-force generating method of step b) is selected from the group consisting of sonication, homogenization, atomization, grinding, jet-milling, and ball-milling.

49. The method of claim 44, wherein the shear-force generating method of step d) is selected from the group consisting of sonication, homogenization, atomization, grinding, jet-milling, or ball-milling.

50. A method of preparing the lipid particle of claim 1 comprising:
a) combining a hydrophobic bioactive agent, an inverted hexagonal phase forming lipid, and an amphiphile in an aqueous solution; and
b) mixing the mixture from step a) by a shear-force generating method at least until a milky suspension forms.

51. The method of claim 50, wherein the suspension from step b) is further fractionated by centrifugation, density gradient centrifugation, or gravitational settlement to obtain particles with a certain size distribution or to remove larger lipid particles.

52. The method of claim 50, wherein the suspension from step b) is further filtered to remove larger lipid particles.

53. The method of claim 50, wherein the suspension from step b) is further fractionated by gel-permeation chromatographic method to obtain particles with a certain size distribution or to remove larger lipid particles.

54. The method of claim 50, wherein the shear-force generating method is selected from the group consisting of sonication, homogenization, atomization, grinding, jet-milling, or ball-milling.

55. A method of preparing the lipid particle of claim 1 comprising:
a) co-dissolving a hydrophobic bioactive agent and an inverted hexagonal phase-forming lipid in an organic solvent;
b) infusing the solution from step a) into an aqueous solution to form a suspension;
c) removing substantially all of the organic solvent from the mixture of step b) to form a second suspension;
d) dissolving an amphiphile in an organic solvent;
e) infusing the solution from step c) into an aqueous solution to form a third suspension;
f) removing substantially all of the organic solvent from the mixture of step d) to form a fourth suspension; and
g) mixing the suspensions from steps c) and f) by a shear-force generating method.

56. The method of claim 55, wherein the suspension from step g) is further fractionated using centrifugation, density gradient centrifugation, or gravitational settlement to obtain particles with a certain size distribution or to remove larger lipid particles.

57. The method of claim 55, wherein the suspension from step g) is further filtered to remove larger lipid particles.

58. The method of claim 55, wherein the suspension from step g) is further fractionated by gel-permeation chromatographic methods to obtain particles with a certain size distribution, or to remove larger lipid particles.

59. The method of claim 55, wherein the shear-force generating method of step g) is selected from the group consisting of sonication, homogenization, atomization, grinding, jet-milling, and ball-milling.

60. A method of aseptically preparing the lipid particle of claim 1 comprising:
a) combining a hydrophobic bioactive agent and an inverted hexagonal phase-forming lipid in a non-aqueous solution;
b) dissolving an amphiphile in a non-aqueous solution;
c) sterile-filtering the solution from step a);
d) sterile-filtering the solution from step b);
e) combining a sterile aqueous solution or sterile water with the sterile-filtered solution from step c) to form a suspension;
f) combining a sterile aqueous solution or sterile water with a sterile-filtered solution from step d) to form a suspension;
g) removing non-aqueous solvent from the suspension of step e) by aseptic evaporation, dialysis, or diafiltration to form an aqueous suspension;
h) removing non-aqueous solvent from the suspension of step f) by aseptic evaporation, dialysis, or diafiltration to form an aqueous suspension;
i) combining the aqueous suspension from step g) and the aqueous suspension from step h); and
j) mixing the mixture from step i) by a shear-force generating method at least until a milky suspension forms.

61. The method of claim 60, wherein the suspension from step j) is further fractionated by centrifugation, density gradient centrifugation, or gravitational settlement to obtain particles with a certain size distribution or to remove larger lipid particles.

62. The method of claim 60, wherein the suspension from step j) is further filtered to remove larger lipid particles.

63. The method of claim 60, wherein the suspension from step j) is further fractionated by gel-permeation chromatographic method to obtain particles with a certain size distribution or to remove larger lipid particles.

64. The method of claim 60, wherein the shear-force generating method of step j) is selected from the group consisting of sonication, homogenization, atomization, grinding, jet-milling, or ball-milling.

65. A method of freeze-drying the lipid particles from claim 1 comprising:
a) adding the lipid particles to a 5% wt/vol solution of cryoprotactant to form a suspension; and
b) vacuum-drying the suspension from step a) at a temperature below 0 ° C. to form vacuum-dried lipid particles.

66. The method of claim 65, wherein the cryoprotactant is lactose.

67. The method of claim 65, wherein the vacuum-dried lipid particles are further treated to form a powder.

68. The method of claim 65, wherein further treatment comprises grinding, ball milling, or jet milling.

69. A method of treating a patient for lung disease comprising administering to the patient a therapeutically effective amount of the lipid particle of any of claim 4, 5, 6, 7, or 8.

70. A kit comprising the lipid particles of claim 1 and instructions for use thereof.
